(19) United States
(12) Patent Application Publication
(10) Pub. No.: US 2025/0218056 A1
(43) Pub. Date: Jul. 3, 2025

(54) GENERATING PICTURE DESCRIPTION TASK IMAGES USING AI

(71) Applicant: International Business Machines Corporation, Armonk, NY (US)

(72) Inventors: Raquel Norel, Bronx, NY (US); Bo Wen, Chappaqua, NY (US); Carla Paola Agurto Rios, Ossining, NY (US); Chen Wang, Chappaqua, NY (US)

(21) Appl. No.: 18/403,128

(22) Filed: Jan. 3, 2024

(51) Int. Cl. G06T 11/00; G06F 40/40; G16H 50/20

(52) U.S. Cl. CPC G06T 11/00; G06F 40/40; G16H 50/20

(57) ABSTRACT

The present inventive concept provides for a method of generating picture description task images using AI. The method includes using AI to generate a story based on a user input prompt and/or a predetermined input. AI is used to generate an image based on the generated story. AI is used to generate written descriptions of the generated image simulating cohorts of healthy individuals and individuals with a predetermined condition. Diagnostic linguistic features are extracted from written descriptions of the cohorts of healthy individuals and individuals with the predetermined condition. The extracted diagnostic linguistic features of the written descriptions for the cohorts of healthy individuals and individuals with the predetermined condition are compared. The generated image is used in a picture description task when the compared extracted features of the written descriptions of the cohorts of healthy individuals and individuals with the predetermined condition exhibit a predetermined threshold of difference.

### BACKGROUND

Exemplary embodiments of the present inventive concept relate to generating picture description task images, and more particularly, to generating picture description task images using AI (Artificial Intelligence).

The quality and richness of information contained in pictures used for picture description tasks directly affect the accuracy and efficacy of diagnoses (e.g., neurological/psychiatric). Traditional methods of picture generation often produce images that lack sufficient detail and complexity, thereby limiting the subject's capacity to provide rich and detailed descriptions. This limitation, in turn, impacts the baseline for downstream psychiatric/neurological analysis and potentially compromises the accuracy of diagnosis.

### SUMMARY

Exemplary embodiments of the present inventive concept relate to a method, a computer program product, and a system for generating picture description task images using AI.

According to an exemplary embodiment of the present inventive concept, a method of generating picture description task images using AI is provided. The method includes using AI to generate a story based on at least one of a user input prompt and a predetermined input. AI is used to generate an image based on the generated story. AI is used to generate written descriptions of the generated image simulating a cohort of healthy individuals and a cohort of individuals with a predetermined condition. Diagnostic linguistic features are extracted from written descriptions of a cohort of healthy individuals and a cohort of individuals with the predetermined condition. The extracted diagnostic linguistic features of the written descriptions for the cohort of healthy individuals and the cohort of individuals with the predetermined condition are compared. The generated image is used in a picture description task when the compared extracted features of the written descriptions of the cohort of healthy individuals and the cohort of individuals with the predetermined condition exhibit at least a predetermined threshold of difference.

According to an exemplary embodiment of the present inventive concept, a computer program product (CPP) for generating picture description task images using AI is provided. The CPP includes one or more computer-readable storage media and program instructions stored on the one or more non-transitory computer-readable storage media capable of performing a method. The method includes using AI to generate a story based on at least one of a user input prompt and a predetermined input. AI is used to generate an image based on the generated story. AI is used to generate written descriptions of the generated image simulating a cohort of healthy individuals and a cohort of individuals with a predetermined condition. Diagnostic linguistic features are extracted from written descriptions of a cohort of healthy individuals and a cohort of individuals with the predetermined condition. The extracted diagnostic linguistic features of the written descriptions for the cohort of healthy individuals and the cohort of individuals with the predetermined condition are compared. The generated image is used in a picture description task when the compared extracted features of the written descriptions of the cohort of healthy individuals and the cohort of individuals with the predetermined condition exhibit at least a predetermined threshold of difference.

According to an exemplary embodiment of the present inventive concept, a computer system (CS) for generating picture description task images using AI is provided. The CS includes one or more computer processors, one or more computer-readable storage media, and program instructions stored on the one or more of the computer-readable storage media for execution by at least one of the one or more processors capable of performing a method. The method includes using AI to generate a story based on at least one of a user input prompt and a predetermined input. AI is used to generate an image based on the generated story. AI is used to generate written descriptions of the generated image simulating a cohort of healthy individuals and a cohort of individuals with a predetermined condition. Diagnostic linguistic features are extracted from written descriptions of a cohort of healthy individuals and a cohort of individuals with the predetermined condition. The extracted diagnostic linguistic features of the written descriptions for the cohort of healthy individuals and the cohort of individuals with the predetermined condition are compared. The generated image is used in a picture description task when the compared extracted features of the written descriptions of the cohort of healthy individuals and the cohort of individuals with the predetermined condition exhibit at least a predetermined threshold of difference.

### BRIEF DESCRIPTION OF THE DRAWINGS

The following detailed description, given by way of example and not intended to limit the exemplary embodiments solely thereto, will best be appreciated in conjunction with the accompanying drawings, in which:

FIG. 1 illustrates a schematic diagram of computing environment 100 including a generating picture description task images using AI program 150, in accordance with an exemplary embodiment of the present inventive concept.

FIG. 2 illustrates a block diagram of components included in the generating picture description task images using AI program 150, in accordance with an exemplary embodiment of the present inventive concept.

FIG. 3 illustrates a flowchart of a method of generating picture description task images using AI 300, in accordance with an exemplary embodiment of the present inventive concept.

It is to be understood that the included drawings are not necessarily drawn to scale/proportion. The included drawings are merely schematic examples to assist in understanding of the present inventive concept and are not intended to portray fixed parameters. In the drawings, like numbering may represent like elements.

### DETAILED DESCRIPTION

Exemplary embodiments of the present inventive concept are disclosed hereafter. However, it shall be understood that the scope of the present inventive concept is dictated by the claims. The disclosed exemplary embodiments are merely illustrative of the claimed system, method, and computer program product. The present inventive concept may be embodied in many different forms and should not be construed as limited to only the exemplary embodiments set forth herein. Rather, these included exemplary embodiments are provided for completeness of disclosure and to facilitate an understanding to those skilled in the art. In the detailed description, discussion of well-known features and techniques may be omitted to avoid unnecessarily obscuring the presented exemplary embodiments.

References in the specification to “one embodiment,” “an embodiment,” “an exemplary embodiment,” etc., indicate that the embodiment described may include a particular feature, structure, or characteristic, but not every embodiment may necessarily include that feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to implement such feature, structure, or characteristic in connection with other embodiments whether explicitly described.

In the interest of not obscuring the presentation of the exemplary embodiments of the present inventive concept, in the following detailed description, some processing steps or operations that are known in the art may have been combined for presentation and for illustration purposes, and in some instances, may have not been described in detail. Additionally, some processing steps or operations that are known in the art may not be described at all. The following detailed description is focused on the distinctive features or elements of the present inventive concept according to various exemplary embodiments.

The proposed inventive concept addresses the aforementioned problems associated with picture description task images by utilizing generative AI to systematically generate high-quality, complex images. These images can be crafted based on detailed text prompts derived from analytic targets, ensuring that each image contains a wealth of information for description. Furthermore, by incorporating AI in the quality control and analysis stages, the pipeline ensures that the produced images meet the necessary quality standards and assists in the comparison of human and AI image descriptions.

Through this system and method, the invention solves the inherent limitations of traditional picture generation for psychiatric diagnoses. It provides a robust, reliable, and efficient solution to create rich and detail-oriented images that enhance the subject's ability to provide in-depth written descriptions, thereby improving the accuracy of psychiatric/neurological diagnoses. The present invention is a novel system and method that employs generative artificial intelligence (AI) to create rich, detailed images for psychiatric/neurological picture description tasks. It can translate an analytic target into a text prompt, which then guides the AI in generating a corresponding image. This image is subsequently described by AI, and the resulting description is compared with the original text prompt for quality control. Finally, the descriptions provided by both a human subject and the AI are compared to the original text prompt, thereby aiding in neurological/psychiatric diagnosis/analysis. This innovative pipeline design revolutionizes the way picture description tasks are conducted in neurological/psychiatric diagnosis. It not only ensures the production of high-quality images that contain abundant information for interpretation, but also introduces an efficient AI-assisted method for quality control and analysis. The present inventive concept significantly enhances the accuracy and reliability of psychiatric/neurologic diagnoses, which are crucial for informing treatment strategies and monitoring patient progress.

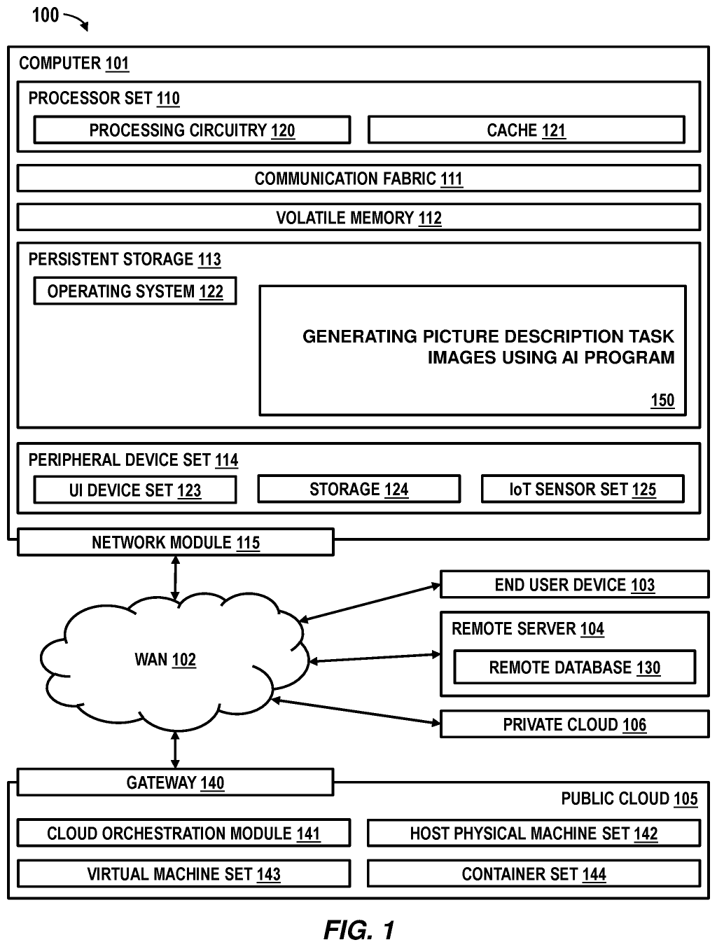

FIG. 1 illustrates a schematic diagram of computing environment 100 including the generating picture description task images using AI program 150, in accordance with an exemplary embodiment of the present inventive concept.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as the generating picture description task images using AI program 150. In addition to block 150, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 150, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 150 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 150 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economics of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

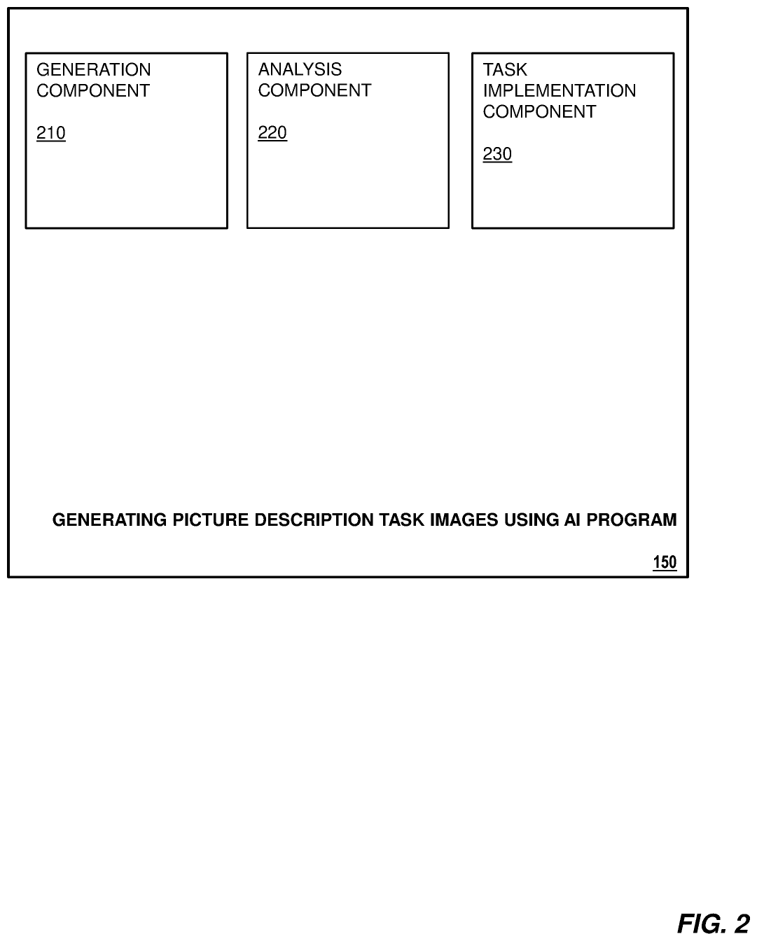

FIG. 2 illustrates a block diagram of components included in the generating picture description task images using AI program 150, in accordance with an exemplary embodiment of the present inventive concept.

The generating picture description task images using AI program 150 can include a generation component 210. The generation component 210 can include a user interactive interface and/or can be connected to various types of AI (e.g., chatbot, natural language processing (NLP), text-to-speech, classification models, large language models (e.g., LLM), computer vision, etc.). The generation component 210 can obtain picture description task multimedia (e.g., audio, video, text, animations, etc.) via user input, analyzed user input prompt, and/or autonomously via the network (e.g., relevant repositories, authorized and/or anonymized provider/entity/patient medical records, internet search, etc.) based on a predetermined condition (e.g., neurological/psychiatric) and/or picture description task. The picture description task multimedia can include: picture description task descriptions, picture description task stories, picture description task images, picture description task responses (e.g., computer vision behavioral analysis, text-to-speech written descriptions, and/or manual written descriptions) from a cohort of healthy individuals and a cohort of individuals with the predetermined condition, picture description task results/discussions in medical literature, etc. The generation component 210 can extract features from the picture description task multimedia, such as: picture description task characteristics (e.g., types, prevalence, accuracy/efficacy/reliability/validity/sensitivity, experimental design, descriptions, disclaimers, symptoms, protocols, parameters, criterion, thresholds, etc.), target predetermined condition/differential diagnostic characteristics (e.g., names, symptoms, parameters, criteria, risk factors, statistical incidence of occurrence/misdiagnosis, comorbidities, hereditability, demographics, progressions, prognosis, etc.), picture description task image/story characteristics (linguistics, tone, style, complexity, syntax, efficacy/accuracy, prevalence, narratives, complexity, character names, character quantities, character emotional states, character ages, character appearances, character relationships, character roles, character titles, character actions, character reactions, character interactions, character backgrounds/experiences, scene descriptions, scene locations, scene day, scene date, scene time, scene season, scene objects, scene events, scene backgrounds, etc.), written description characteristics (e.g., linguistics, tone, style, syntax, complexity, coherence, accuracy, length, time, vocabulary, emphases, omissions, characterizations, etc.) and/or user characteristics (e.g., age, demographics, family history, background/experiences, diagnoses, non-diagnoses, differential diagnoses, complaints, progressions, presentations, medical history, medical appointment frequency/type, etc.).

The generation component 210 can be trained to perform picture description related classification tasks (e.g., new written description predetermined condition diagnosis/non-diagnosis/differential diagnosis, generate simulated written descriptions/stories/prompts/images, etc.) using analyzed picture description task multimedia related to at least one predetermined condition via supervised and/or unsupervised learning.

The generation component 210 can generate at least one picture description task story based on at least one user input prompt, predetermined input (e.g., picture description task, accuracy/efficacy/reliability/validity/sensitivity, condition and/or symptoms, keyword, summary, extracted features, etc.) and/or network search (e.g., supplemented research). The generation component 210 can generate at least one picture description task image and/or written description prompt based on the generated picture description task story. In the case of a plurality of potential generated images, stories, and/or written description prompts, the generation component 210 can present descriptions and/or options (e.g., different narratives, complexities, relative diagnostic advantages/disadvantages, etc.) to the user for selection. The generation component 210 can generate simulated written descriptions for a cohort of healthy individuals and a cohort of unhealthy individuals and extract features therefrom. Based on the extracted features and/or the predetermined input, the generation component 210 can generate a story, image, written description prompts, and/or simulated written descriptions that are predicted to elicit/illustrate distinguishable written description characteristics of the story as between the cohort of healthy individuals and the cohort of individuals with the predetermined condition.

### For Example

### Provider User Prompt to Generation Component 210:

Can you assist in brainstorming several scene descriptions for creating an image for cognitive assessment? The scenarios should involve multiple characters (2-5) who can engage in interaction with the environment. For example, one character could be distracted, leading to humorous or negative outcomes such as a water spill or a fall, or a surprising event. The scene must include several objects in the environment that characters can manipulate or interact with. The depiction needs to offer stimuli that can be interpreted and narrated. The location, such as a beach, pool, or highway, plays a crucial role as well.

### Generation Component 210 Responses:

Certainly! Here are several scene descriptions for creating an image for cognitive assessment with multiple characters and interactive environments:

Location: A busy supermarket. Description: A group of friends is grocery shopping together. One character gets distracted by a display of free samples and accidentally bumps into a stacked pyramid of canned goods, causing them to collapse. The other characters react with surprise and amusement as the cans roll around the aisle.

Location: A crowded amusement park. Description: A family of five rides a roller coaster together. As the coaster reaches its peak, one character's hat flies off and lands on the head of a nearby statue, surprising everyone. The family members point and laugh while the character tries to retrieve the hat.

Location: A neighborhood park with a pond. Description: A group of friends gathers around a picnic table near the pond, engrossed in a card game. One character, engrossed in the game, accidentally knocks over a cup of juice, spilling it onto the cards. The other characters react with surprise and frustration, trying to salvage the game.

Location: A scenic hiking trail. Description: A hiking group of friends is exploring a beautiful trail. One character, distracted by taking pictures, accidentally steps into a patch of mud and slips, falling onto their backside. The other characters rush to help them up while laughing at the unexpected mishap.

Location: A beach volleyball court. Description: Two teams are playing an intense match of beach volleyball. One character, distracted by a seagull flying overhead, loses focus and misses a crucial shot. The other characters react with surprise as the ball lands far from its intended target.

Location: A bustling city square. Description: A group of friends is sitting on a bench, enjoying the atmosphere. One character's ice cream cone starts melting rapidly due to the hot weather, causing it to drip onto their hand. They react with surprise and try to lick the melting ice cream quickly.

Location: A bowling alley. Description: A group of friends is having a friendly bowling competition. One character, distracted by a flashy advertisement on the electronic score display, forgets to release the bowling ball and stumbles forward, narrowly avoiding a fall. The other characters react with surprise and laughter.

Remember, you can modify and adapt these descriptions to suit your specific needs, and feel free to incorporate different elements from various scenes to create unique scenarios.

### Provider User Input Prompt:

Every one of these situations is characterized by several individuals and objects, and incorporates a range of elements such as diversion, astonishment, and interaction, all taking place in unique locations.

### Features for Alzheimer's Disease (AD) and Schizophrenia:

For AD, we'll be assessing aspects like verbosity, lexical richness via total word count, unique word use, utterance number, and even character count in words, as they can indicate cognitive aging. Common features in dementia, such as repetitiveness, will also be considered. We'll evaluate Information Content Units (ICU) to quantify object or action count in an image. Word frequency will be considered, as individuals with AD tend to use predetermined words more frequently. A characteristic difficulty in forming large and complex syntactic sentences will also be assessed.

With respect to Schizophrenia, we'll examine aspects like a reduction in syntax complexity and impaired semantics, evident in the organization of singular propositions into larger structures. Changes in parts of speech, such as increased use of pronouns but decreased use of adverbs, adjectives, and determiners (e.g., “the,” “a,”), will also be assessed.

### For Schizophrenia:

In schizophrenia, we will look for reduced syntactic complexity. We will also pay attention to problems with semantics, such as difficulties in organizing individual propositions into larger units of meaning. Changes in parts of speech will also be noted, such as increased use of pronouns but fewer adverbs, adjectives, and determiners (for example, “the,” “a”).

In individuals with schizophrenia, we'll be evaluating factors such as a decrease in complexity of sentence structure. We will also observe any disruptions in semantics, like issues in compiling individual statements into broader constructs. Shifts in speech parts, like more usage of pronouns but less of adverbs, adjectives, and determiners (such as “the,” “a”), will also be noted.

For those with schizophrenia, we're on the lookout for simplified sentence structures. Impaired semantics, seen in difficulties forming individual ideas into broader structures, will also be considered. We will also note changes in language use, such as a preference for pronouns but a scarcity of adverbs, adjectives, and determiners (e.g., “the,” “a”).

### Provider User Input Prompt:

Can you simulate how an AD patient will describe the first scene?

### Generation Component 210 Response:

Certainly! Here's a simulation of how an AD patient might describe the first scene, considering the features associated with AD:

“The coffee shop, yes, the coffee shop was crowded. There were people everywhere, talking, talking so much. I sat at a table, yes, with a laptop. I was working, typing, typing lots of words. Then, oh no, I reached for my coffee cup but knocked it, spilled it. It went everywhere, on my laptop, on the table. Hot, it was hot! Too many words, so many words, but now, oh, the laptop doesn't work. The people looked, they looked at me. Embarrassed, I was embarrassed. So many words, but now, just a mess.”

In this simulation, you can see characteristics of verbosity, repetition, and difficulties with lexical richness and syntactic complexity. The narrative includes a focus on the crowded environment, the character's actions, and the negative outcome, but with reduced coherence and repetition of certain phrases.

### Provider User Input Prompt:

Can you help me come up with a few scene descriptions for making a picture for cognitive tests? The scene should have: Several characters (2-5) (so they can interact). Usually one character is distracted, so something funny/bad happens (water spilling, someone falling) or there is a surprise element. There should be several objects that characters can interact with. The scene must provide stimuli so histories can be described. Location also is important (e.g., beach, pool, highway, etc.).

### Generation Component 210 Response:

Sure, I'd be happy to help you with that. Here are a few scene descriptions that could potentially be used for your cognitive tests:

Picnic in the Park: Four friends are having a picnic in a lush, green park. One of them is busy looking at a bird in the tree, oblivious to the fact that a squirrel is sneaking up on their picnic basket. The other friends are laughing and pointing at the squirrel, while another friend is about to sit on a sandwich that someone has dropped. There are various objects around like a frisbee, a picnic blanket, a thermos of coffee, and a kite stuck in a tree.

Beach Day: A family of five is spending the day at the beach. The dad is building a sandcastle with the youngest child, but he's so engrossed in it that he doesn't realize the tide is coming in and about to wash it away. The mom is reading a book, and her ice cream is melting in the sun. The two older children are playing with a beach ball, which is about to hit a sleeping sunbather. Around them are beach towels, buckets and spades, seashells, and a cooler box.

Ski Lodge Mishap: Three friends are in a cozy ski lodge. One is looking out the window at the beautiful snowfall, distractedly pouring hot chocolate all over the table instead of into his cup. Another friend is laughing and about to take a picture, while the third is slipping on a puddle of the spilled hot chocolate. Scattered around the room are ski gear, a crackling fireplace, board games, and mugs of hot drinks.

Roadside Repair: Two friends are on a highway, trying to change a flat tire on their car. One is so busy talking on the phone and giving instructions that he doesn't notice the other one struggling with the jack, which is about to slip. The car trunk is open, revealing a suitcase, a map, a cooler, and a spare tire. A signpost nearby shows that they're only a few miles away from a city.

Swimming Pool Party: A pool party is taking place with four friends. One is so busy setting up the music player that he's not noticed he's about to step into the pool. A second friend is inflating a giant pool float, and a third is grilling hot dogs, unaware that one has fallen off the grill. The fourth friend is about to dive into the pool, oblivious to the pool float being inflated in his path. The scene is filled with summer decorations, pool toys, BBQ equipment, and garden furniture.

Each of these scenarios involves multiple characters and objects, with various elements of distraction, surprise, and interaction, as well as unique locations.

### Provider User Input Prompt:

Here are features that we will be looking for in patients with AD and Schizophrenia. Can you simulate how a Schizophrenia patient will be describing the first scene? Here is a list of features for AD besides the ones listed above: verbosity and lexical richness measured with total number of words, unique words, number of utterance or even number of characters in words, are predictors of cognitive aging. Repetitiveness is very common in dementia Information content units (ICU) for example to quantify the number of objects or actions in a picture. Word frequency: depending of the word, people with AD tend to use some words more often than others. AD people have difficulty to use large and syntactically complex sentences. Here is for Schizophrenia besides the listed above: Reduction in syntax complexity. Impaired semantics, such as the organization of individual propositions into larger structures. Part of speech, e.g., used more pronouns but fewer adverbs, adjectives, and determiners (e.g., “the,” “a,”).

### Generation Component 210 Response:

Absolutely, I can help with that. Here's an example of how a patient with schizophrenia might describe the first scene, the “Picnic in the Park”. This description attempts to reflect some of the language characteristics you mentioned, including reduced syntactic complexity, impaired semantics, and altered use of parts of speech:

“Friends. There are four. In a park. Green around. One looking at a bird. Bird in a tree. Squirrel near them. Sneaking, yes, sneaking. Friends laugh. Point at squirrel. Another friend, he's going to sit. On a sandwich. Sandwich on the ground. Picnic. Items around. Frisbee there. Blanket on ground. Coffee in a thermos. Kite. It's stuck. Stuck in tree.”

Please note that this is a generalized depiction and may not accurately represent every individual with schizophrenia, as the symptoms and their severity can vary widely from person to person. Similarly, the specific language deficits can vary based on individual differences and the course of the disease.

The generating picture description task images using AI program 150 can include an analysis component 220. The analysis component 220 can compare the simulated written descriptions (e.g., extracted features and/or distributions) from the cohort of healthy individuals and the cohort of unhealthy individuals. The analysis component 220 can determine whether the compared simulated written descriptions (e.g., extracted features and/or distributions) are within a predetermined threshold of difference sufficient to render a diagnosis in the cohort of individuals with the predetermined condition (e.g., within a predetermined threshold of at least one of accuracy, precision, validity, sensitivity, reliability, etc.). The analysis component 220 can approve the generated image for use in a picture description task (e.g., diagnosis/non-diagnosis of the predetermined condition and/or differential diagnosis) and/or store the user input prompt, predetermined input, picture description task/image/story/written descriptions/extracted features in a repository when the compared extracted features of the written descriptions of the cohort of healthy individuals and the cohort of individuals with the predetermined condition exhibit at least the predetermined threshold of difference. The analysis component 220 can tune at least one of the predetermined input, the generated story, the generated image, the generated written description prompt, and the generated simulated written descriptions automatically or via user solicitation when the compared extracted features of the written descriptions of the cohort of healthy individuals and the cohort of individuals with the predetermined condition do not exhibit at least the predetermined threshold of difference. In an embodiment, the analysis component 220 can make modifications to learned experimental designs of the picture description tasks predicted to improve distinguishing comparisons of the written descriptions (e.g., extracted features and/or distributions) within the predetermined threshold of difference sufficient to render the diagnosis in the cohort of individuals with the predetermined condition within the predetermined threshold (e.g., accuracy, precision, validity, sensitivity, reliability, etc.).

For example, the analysis component 220 can compare the generated simulated written descriptions from the cohort of healthy individuals and the cohort of individuals with AD and/or Schizophrenia. The analysis component 220 determines that the simulated written descriptions demonstrate inconsistent accuracy over iterations, thus the analysis component increases characters and actions occurring in the story and corresponding image until a threshold of reliability and accuracy is achieved.

The generating picture description task images using AI program 150 can include a task implementation component 230. The task implementation component 230 can be displayed on the interactive interface for users (e.g., research scientists, providers, patients, etc.). The task implementation component 230 can select (e.g., generate anew and/or retrieve from the repository) a picture description task (e.g., experimental design, story, image, written prompts, simulated written descriptions, etc.) based on the user input prompt and/or the predetermined input by a user (e.g., research scientist and/or provider). The task implementation component 230 can administer the selected picture description task with a user (e.g., patient) via the interactive interface. The task implementation component 230 can provide written descriptions prompts and/or extract features and/or distributions from the responsive written descriptions and compare them with at least one of the simulated/actual written descriptions from the cohort of healthy individuals and the cohort of individuals with the predetermined condition. The task implementation component 230 can render a diagnosis/non-diagnosis/differential diagnosis and descriptions thereof, provide a confidence level, and/or confounding factors, etc. When the diagnosis is unexpected (e.g., discrepancy between provider diagnosis and/or diagnosis associated with extracted features from the patient characteristics), the task implementation component 230 can perform at least one other picture description task and/or modification automatically and/or upon user approval. The task implementation component 230 can tune (e.g., weights, qualities, quantities, etc.) of at least one of the predetermined input, the generated story (e.g., increase/decrease details, complexity, length, etc.), the generated image, the generated written description prompt, and/or the generated simulated written descriptions when the compared extracted features of the written descriptions of the cohort of healthy individuals and the cohort of individuals with the predetermined condition do not exhibit at least the predetermined threshold of difference and/or do not meet the predetermined thresholds of at least one of accuracy, precision, validity, sensitivity, and/or reliability. The task implementation component 230 can provide the user with the diagnosis and corresponding evaluations/scores via the interactive interface with annotations/descriptions. In an embodiment, the task implementation component can provide user surveys to confirm diagnoses/non-diagnoses and/or probe for differential diagnoses.

For example, the task implementation component 230 uses the provider input prompts, predetermined inputs, generated stories/images/written description prompts/written descriptions that exhibited the predetermined thresholds of compared difference. The task implementation component 230 administers the picture description task for AD and/or Schizophrenia to a new patient user. The new patient user is presented with a written description prompt related to the crowded amusement park and the corresponding approved image. The new patient written description reads: “A bustling city square. Description: A group of friends is sitting on a bench, it's a relief from constant loneliness. They are enjoying the atmosphere since it usually rains. One character's ice cream cone starts melting rapidly due to the hot weather, causing it to drip onto their hand, typical. The friends react with surprise and ridicule and try to lick the melting ice cream quickly before it makes an even bigger mess.” Although the compared differences in the new patient written description exhibit the predetermined threshold of difference from the cohort of healthy individuals and excluded Schizophrenia, the tone of the new patient written description suggests a potential differential diagnosis of depression, which is confounded by overall similarities in characteristic written description features. The task implementation component 230 then administers the picnic in the park picture description task. The new patient written description is as follows: “The coffee shop, yes, the coffee shop was overcrowded. There were people everywhere, talking, talking too much. Very loud. I sat at a table, yes, with a laptop. I was working, typing, typing lots of words. Then, oh no, I reached for my coffee cup but knocked it, spilled it. Typical! It went everywhere, on my laptop, on the table. Hot, it was hot! Ruined nice shirt. Too many words, so many words, but now, oh, the laptop doesn't work. The people looked, they looked at me. Embarrassed, I was embarrassed and sad. So many words, but now, just a mess. Like everything else.” The task implementation component 230 confirms a diagnosis of depression within a 90% degree of confidence and presents the conclusion to the awaiting provider.

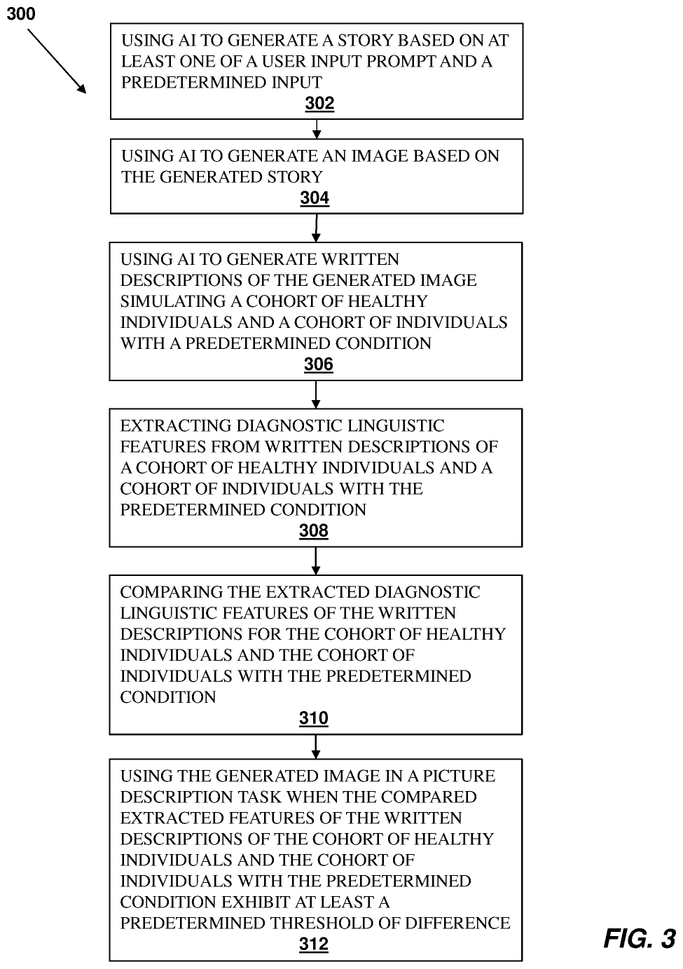

FIG. 3 illustrates a flowchart of the method of generating picture description task images using AI 300, in accordance with an exemplary embodiment of the present inventive concept.

The method of generating picture description task images using AI can include:
using AI to generate a story based on at least one of a user input prompt and a predetermined input (step 302);
using AI to generate an image based on the generated story (step 304);
using AI to generate written descriptions of the generated image simulating a cohort of healthy individuals and a cohort of individuals with a predetermined condition (step 306);
extracting diagnostic linguistic features from written descriptions of a cohort of healthy individuals and a cohort of individuals with the predetermined condition (step 308);
comparing the extracted diagnostic linguistic features of the written descriptions for the cohort of healthy individuals and the cohort of individuals with the predetermined condition (step 310); and
using the generated image in a picture description task when the compared extracted features of the written descriptions of the cohort of healthy individuals and the cohort of individuals with the predetermined condition exhibit at least a predetermined threshold of difference (step 312).

Based on the foregoing, a computer system, method, and computer program product have been disclosed. However, numerous modifications, additions, and substitutions can be made without deviating from the scope of the exemplary embodiments of the present inventive concept. Therefore, the exemplary embodiments of the present inventive concept have been disclosed by way of example and not by limitation.

What is claimed is:

1. A method of generating picture description task images using AI, the method comprising:
using AI to generate a story based on at least one of a user input prompt and a predetermined input;
using AI to generate an image based on the generated story;
using AI to generate written descriptions of the generated image simulating a cohort of healthy individuals and a cohort of individuals with a predetermined condition;
extracting diagnostic linguistic features from written descriptions of a cohort of healthy individuals and a cohort of individuals with the predetermined condition;
comparing the extracted diagnostic linguistic features of the written descriptions for the cohort of healthy individuals and the cohort of individuals with the predetermined condition; and
using the generated image in a picture description task when the compared extracted features of the written descriptions of the cohort of healthy individuals and the cohort of individuals with the predetermined condition exhibit at least a predetermined threshold of difference.

2. The method of claim 1, wherein the using the generated image in a picture description task includes approving the generated image for diagnostic use of the predetermined condition.

3. The method of claim 1, wherein the using the generated image in a picture description task includes outputting a diagnosis or non-diagnosis for the predetermined medical condition.

4. The method of claim 1, further comprising:
generating simulated written descriptions for at least one individual from at least one of the cohort of healthy individuals and the cohort of individuals with the predetermined condition.

5. The method of claim 1, further comprising:
generating a written description prompt related to the generated image.

6. The method of claim 5, wherein at least one of the generated story, the generated image, and the generated written description prompt are designed to elicit written descriptions from the cohort of healthy individuals and the cohort of individuals with the predetermined condition with compared extracted diagnostic linguistic features that exhibit at least the predetermined threshold of difference.

7. The method of claim 5, further comprising:
tuning at least one of the predetermined input, the generated story, the generated image, the generated written description prompt, and the generated simulated written descriptions when the compared extracted features of the written descriptions of the cohort of healthy individuals and the cohort of individuals with the predetermined condition do not exhibit at least the predetermined threshold of difference.

8. A computer program product (CPP) for generating picture description task images using AI, the CPP comprising:
one or more computer-readable storage media and program instructions stored on the one or more non-transitory computer-readable storage media capable of performing a method, the method comprising:
using AI to generate a story based on at least one of a user input prompt and a predetermined input;
using AI to generate an image based on the generated story;
using AI to generate written descriptions of the generated image simulating a cohort of healthy individuals and a cohort of individuals with a predetermined condition;
extracting diagnostic linguistic features from written descriptions of a cohort of healthy individuals and a cohort of individuals with the predetermined condition;
comparing the extracted diagnostic linguistic features of the written descriptions for the cohort of healthy individuals and the cohort of individuals with the predetermined condition; and
using the generated image in a picture description task when the compared extracted features of the written descriptions of the cohort of healthy individuals and the cohort of individuals with the predetermined condition exhibit at least a predetermined threshold of difference.

9. The CPP of claim 8, wherein the using the generated image in a picture description task includes approving the generated image for diagnostic use of the predetermined condition.

10. The CPP of claim 8, wherein the using the generated image in a picture description task includes outputting a diagnosis or non-diagnosis for the predetermined medical condition.

11. The CPP of claim 8, further comprising:
generating simulated written descriptions for at least one individual from at least one of the cohort of healthy individuals and the cohort of individuals with the predetermined condition.

12. The CPP of claim 8, further comprising:
generating a written description prompt related to the generated image.

13. The CPP of claim 12, wherein at least one of the generated story, the generated image, and the generated written description prompt are designed to elicit written descriptions from the cohort of healthy individuals and the cohort of individuals with the predetermined condition with compared extracted diagnostic linguistic features that exhibit at least the predetermined threshold of difference.

14. The CPP of claim 12, further comprising:
tuning at least one of the predetermined input, the generated story, the generated image, the generated written description prompt, and the generated simulated written descriptions when the compared extracted features of the written descriptions of the cohort of healthy individuals and the cohort of individuals with the predetermined condition do not exhibit at least the predetermined threshold of difference.

15. A computer system (CS) for generating picture description task images using AI, the CS comprising:
one or more computer processors, one or more computer-readable storage media, and program instructions stored on the one or more of the computer-readable storage media for execution by at least one of the one or more processors capable of performing a method, the method comprising:
using AI to generate a story based on at least one of a user input prompt and a predetermined input;
using AI to generate an image based on the generated story;
using AI to generate written descriptions of the generated image simulating a cohort of healthy individuals and a cohort of individuals with a predetermined condition;
extracting diagnostic linguistic features from written descriptions of a cohort of healthy individuals and a cohort of individuals with the predetermined condition;
comparing the extracted diagnostic linguistic features of the written descriptions for the cohort of healthy individuals and the cohort of individuals with the predetermined condition; and
using the generated image in a picture description task when the compared extracted features of the written descriptions of the cohort of healthy individuals and the cohort of individuals with the predetermined condition exhibit at least a predetermined threshold of difference.

16. The CS of claim 15, wherein the using the generated image in a picture description task includes approving the generated image for diagnostic use of the predetermined condition.

17. The CS of claim 15, wherein the using the generated image in a picture description task includes outputting a diagnosis or non-diagnosis for the predetermined medical condition.

18. The CS of claim 15, further comprising:
generating simulated written descriptions for at least one individual from at least one of the cohort of healthy individuals and the cohort of individuals with the predetermined condition.

19. The CS of claim 15, further comprising:
generating a written description prompt related to the generated image.

20. The CS of claim 19, wherein at least one of the generated story, the generated image, and the generated written description prompt are designed to elicit written descriptions from the cohort of healthy individuals and the cohort of individuals with the predetermined condition with compared extracted diagnostic linguistic features that exhibit at least the predetermined threshold of difference.
